(19) United States
(12) Patent Application Publication
(10) Pub. No.: US 2017/0311877 A1
(43) Pub. Date: Nov. 2, 2017

(54) BLOOD-VESSEL RECOGNITION DEVICE AND SURGICAL TREATMENT DEVICE

(71) Applicant: OLYMPUS CORPORATION, Tokyo (JP)

(72) Inventors: Takeshi WATANABE, Tokyo (JP); Shintaro TAKAHASHI, Tokyo (JP); Makoto IGARASHI, Tokyo (JP)

(73) Assignee: OLYMPUS CORPORATION, Tokyo (JP)

(21) Appl. No.: 15/650,144

(22) Filed: Jul. 14, 2017

Related U.S. Application Data
(63) Continuation of application No. PCT/JP2016/051930, filed on Jan. 22, 2016.

(30) Foreign Application Priority Data
Jan. 23, 2015 (JP) PCT/JP2015/051760

(51) Int. Cl. A61B 5/00; A61B 1/00

(52) U.S. Cl. CPC A61B 5/489; A61B 1/00; A61B 5/0075

(57) ABSTRACT

A surgical treatment device is provided with: an action portion; a light emitting part that radiates laser light onto living tissue; a light receiving part that receives scattered light of the laser light scattered by the living tissue; a light detection unit that detects the intensity of the scattered light received by the light receiving part; a frequency analysis unit that obtains time-series data indicating a temporal change in the intensity of the scattered light detected by the light detection unit, to extract an amount of frequency spectrum shift of the scattered light, included in the time-series data; and a determination unit that determines a feature of a blood vessel in the living tissue on the basis of the amount of frequency spectrum shift.

### CROSS-REFERENCE TO RELATED APPLICATIONS

This application is a Continuation Application of International Application No. PCT/JP2016/051930 filed on Jan. 22, 2016, which claims priority to International Application No. PCT/JP2015/051760 filed on Jan. 23, 2015. The contents of International Applications No. PCT/JP2016/051930 and No. PCT/JP2015/051760 are hereby incorporated herein by reference in their entirety.

### TECHNICAL FIELD

The present invention relates to a blood-vessel recognition device and a surgical treatment device.

### BACKGROUND ART

In surgical treatment of living tissue, it is important for a surgeon to accurately recognize the existence of a blood vessel hidden in the inside of the living tissue and to perform treatment so as to avoid the blood vessel. Thus, surgical treatment devices having a function for optically detecting a blood vessel existing in living tissue have been proposed (for example, see PTL 1). In PTL 1, the amount of blood in the living tissue is measured, and it is determined whether a blood vessel exists on the basis of the measured amount of blood.

### CITATION LIST

### Patent Literature

{PTL 1} Publication of Japanese Patent No. 4490807

### SUMMARY OF INVENTION

According to a first aspect, the present invention provides a blood-vessel recognition device including: a light emitting part that radiates laser light onto living tissue; a light receiving part that receives scattered light of the laser light scattered by the living tissue; a light detection unit that detects intensity of the scattered light received by the light receiving part; a frequency analysis unit that analyzes time-series data indicating a temporal change in the intensity of the scattered light detected by the light detection unit, to extract an amount of frequency spectrum shift of the scattered light, the amount of frequency spectrum shift being included in the time-series data; and a determination unit that determines a feature of a blood vessel in the living tissue on the basis of the amount of frequency spectrum shift extracted by the frequency analysis unit.

### BRIEF DESCRIPTION OF DRAWINGS

FIG. 1 is a view showing the overall configuration of a surgical treatment device according to a first embodiment of the present invention.

FIG. 2 is a view for explaining scattering of laser light due to static components in living tissue.

FIG. 3 is a view for explaining scattering of laser light due to dynamic components in the living tissue.

FIG. 4 shows example time-series data of the intensity of scattered light, obtained in a determination unit shown in FIG. 1.

FIG. 5 shows an example Doppler spectrum obtained in the determination unit shown in FIG. 1.

FIG. 6 is a graph showing the relationship between the velocity of a blood flow and the average frequency of a Doppler spectrum.

FIG. 7 is a view showing the overall configuration of a surgical treatment device according to a second embodiment of the present invention.

FIG. 8 is a view for explaining the operation of the surgical treatment device shown in FIG. 7.

FIG. 9 is a partial configuration view showing a modification of the surgical treatment device shown in FIG. 7.

FIG. 10 is a view showing the overall configuration of a blood-vessel recognition device according to a third embodiment of the present invention.

FIG. 11A is a view showing an energy treatment tool to which the blood-vessel recognition device shown in FIG. 10 is attached.

FIG. 11B is a sectional view of an energy treatment tool and the blood-vessel recognition device shown in FIG. 11A, along the line XI-XI.

FIG. 12 is a view showing a modification of the energy treatment tool shown in FIGS. 11A and 11B.

FIG. 13 is a view for explaining the operation of modifications of an irradiation optical fiber and a light-receiving optical fiber.

FIG. 14 is a graph showing a simulation result of a spatial intensity change in scattered light produced in a blood vessel.

FIG. 15 is a graph showing the integrated intensity of scattered light obtained from the graph shown in FIG. 14.

### DESCRIPTION OF EMBODIMENTS

### First Embodiment

A surgical treatment device 100 according to a first embodiment of the present invention will be described below with reference to the drawings.

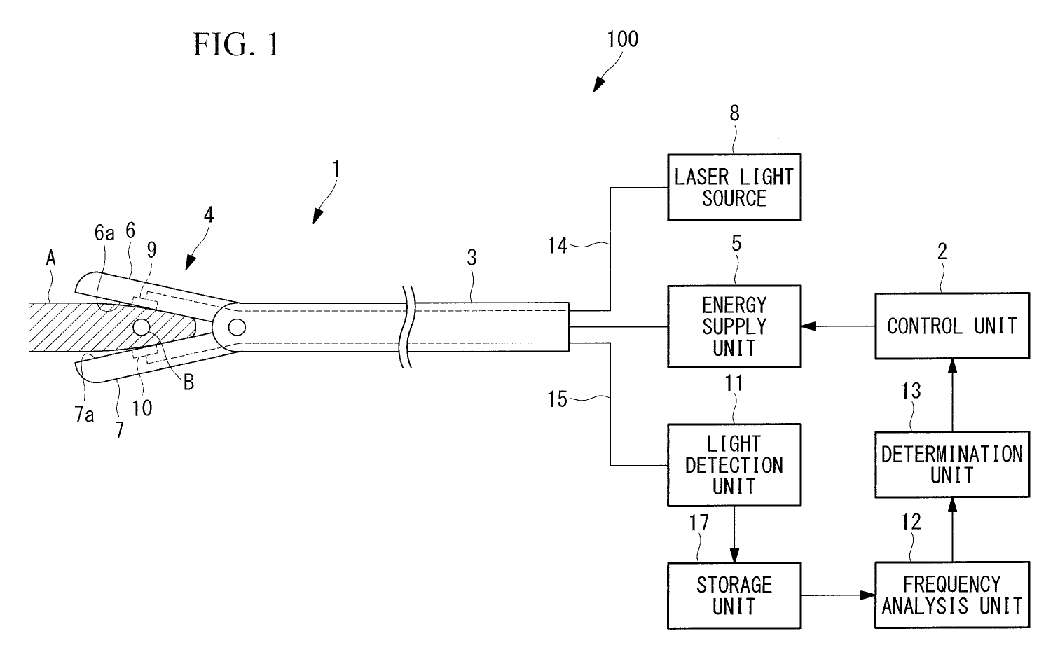

As shown in FIG. 1, the surgical treatment device 100 of this embodiment is provided with: an energy treatment tool 1 with which living tissue A is treated; a blood-vessel detection means that optically detects a blood vessel B in the living tissue A; and a control unit 2 that controls the energy treatment tool 1 on the basis of a detection result obtained by the blood-vessel detection means.

The energy treatment tool 1 is provided with: an elongated shaft portion 3 that can be inserted into the body; an energy action portion 4 that is provided at a distal end of the shaft portion 3 and that causes energy to act on the living tissue A; and an energy supply unit 5 that is connected to a proximal end of the shaft portion 3, for supplying an energy source to the energy action portion 4 via a wire passing through the inside of the shaft portion 3.

The energy action portion 4 is energy forceps that have a pair of jaws 6 and 7 capable of gripping the living tissue A. The upper jaw 6 and the lower jaw 7 have inner surfaces 6a and 7a facing each other. When an energy source (for example, high-frequency current) is supplied from the energy supply unit 5, the upper jaw 6 and the lower jaw 7 produce energy (for example, high-frequency current or ultrasound waves), and the produced energy is emitted from the inner surfaces 6a and 7a toward the living tissue A between the inner surfaces 6a and 7a.

The energy action portion 4 has, as operation modes, an incision mode in which the living tissue A is incised with high energy and a coagulation mode in which the living tissue B is coagulated with low energy that is lower than the high energy in the incision mode. The energy action portion 4 switches between the incision mode and the coagulation mode according to the intensity of the energy source supplied from the energy supply unit 5.

The blood-vessel detection means is provided with: a laser light source 8 that outputs laser light L; a light emitting part 9 that is provided on the inner surface 6a of the upper jaw 6 and that emits the laser light L supplied from the laser light source 8; an irradiation optical fiber (first transmission path) 14 that transmits the laser light L from the laser light source 8 to the light emitting part 9; a light receiving part 10 that is provided on the inner surface 7a of the lower jaw 7 and that receives scattered light S of the laser light L scattered by the living tissue A; a light detection unit 11 that detects the scattered light S received by the light receiving part 10; a light-receiving optical fiber (second transmission path) 15 that transmits the scattered light S from the light receiving part 10 to the light detection unit 11; a storage unit 17 that accumulates data on the intensity of the scattered light S detected by the light detection unit 11; a frequency analysis unit 12 that applies frequency analysis to the data accumulated in the storage unit 17; and a determination unit 13 that determines the presence or absence of a detection-target blood vessel that has a predetermined diameter of blood vessel, on the basis of a frequency analysis result obtained by the frequency analysis unit 12.

The laser light source 8 outputs laser light L in a wavelength range (for example, near-infrared range) that is less absorbed by blood. The laser light source 8 is connected to the light emitting part 9 via the irradiation optical fiber 14, which passes through the inside of the shaft portion 3. The laser light L introduced to the irradiation optical fiber 14 from the laser light source 8 is guided to the light emitting part 9 by the irradiation optical fiber 14 and is emitted from the light emitting part 9 toward the inner surface 7a of the lower jaw 7.

The light receiving part 10 is connected to the light detection unit 11 via the light-receiving optical fiber 15, which passes through the inside of the shaft portion 3. The scattered light S received by the light receiving part 10 is guided to the light detection unit 11 by the light-receiving optical fiber 15 and enters the light detection unit 11.

The light detection unit 11 converts the intensity of the scattered light S introduced from the light-receiving optical fiber 15 into digital values and sequentially sends such digital values to the storage unit 17.

The storage unit 17 stores digital values received from the light detection unit 11 in a time series manner, thereby generating time-series data indicating a temporal change in the intensity of the scattered light S.

The frequency analysis unit 12 periodically obtains the time-series data from the storage unit 17, applies a fast Fourier transform to the obtained time-series data, and calculates the average frequency of the obtained Fourier spectrum.

Here, the time-series data and the Fourier spectrum will be described.

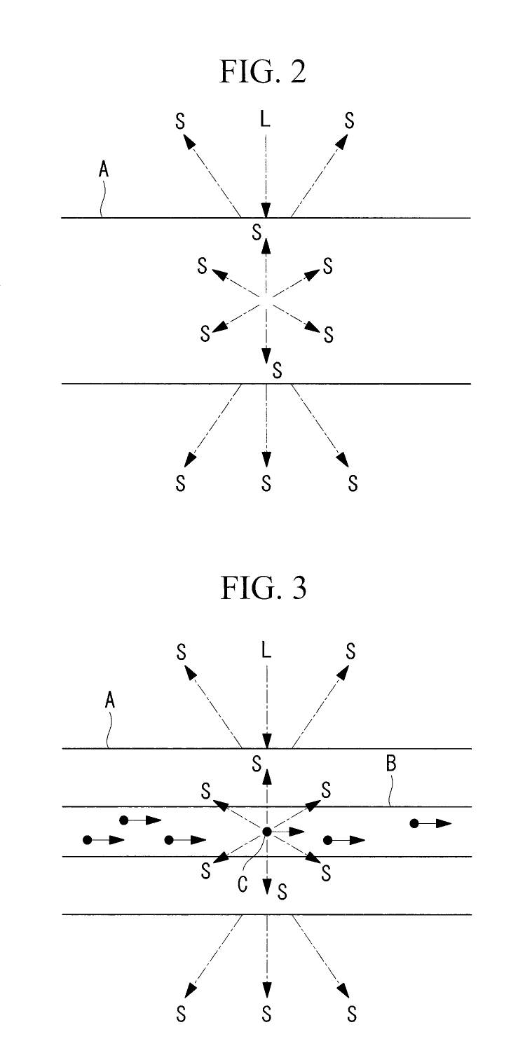

As shown in FIGS. 2 and 3, the living tissue A includes static components that are static, such as fat and leaking blood exposed from the blood vessel B through bleeding, and dynamic components that are moving, such as red blood cells C in blood that moves in the blood vessel B. When the laser light L having a frequency f is radiated onto the static components, scattered light S having the same frequency f as the laser light L is produced. On the contrary, when the laser light L having the frequency f is radiated onto the dynamic components, scattered light S having a frequency f+Δf that is shifted from the frequency f of the laser light L due to the Doppler shift is produced. The amount of frequency spectrum shift Δf at this time depends on the velocity of movement of the dynamic components.

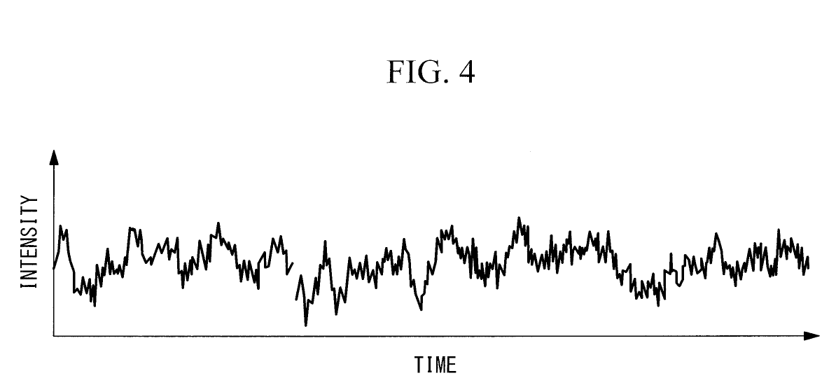

Therefore, when the blood vessel B is included in an area irradiated with the laser light L in the living tissue A, the light receiving part 10 simultaneously receives the scattered light S that is scattered by the blood in the blood vessel B, thus having the frequency f+Δf, and the scattered light S that is scattered by the static components other than the blood in the blood vessel B, thus having the frequency f. As a result, as shown in FIG. 4, the time-series data shows a beat in which the intensity of the scattered light S as a whole changes at Δf due to the interference of the scattered light S having the frequency f and the scattered light S having the frequency f+Δf.

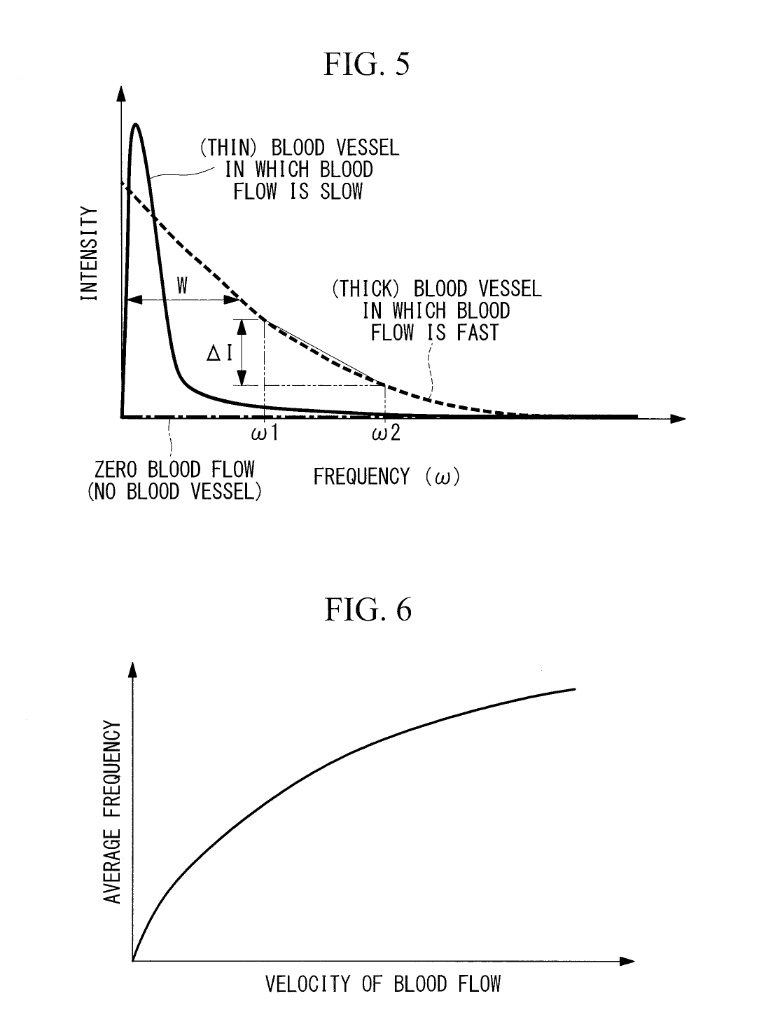

Because the laser light L that has been radiated onto the living tissue A undergoes multiple scattering at the static components and the dynamic components, when the laser light L is incident on the red blood cells, the incident angle formed by the direction of travel of the laser light L and the direction of movement of the red blood cells (the direction of blood flow) is not a single angle but forms a distribution. Thus, the amount of frequency spectrum shift Δf due to the Doppler shift forms a distribution. Therefore, the beat of the intensity of the scattered light S as a whole is obtained by superposition of multiple frequency components in accordance with the distribution of Δf. Furthermore, to be exact, a beat due to the interference of scattered light beams having different frequency shifts is also superimposed. Furthermore, the distribution of Δf expands toward the high frequency side as the blood flow velocity becomes high. When such time-series data is subjected to a fast Fourier transform, as shown in FIG. 5, a Doppler spectrum having the intensity at a frequency ω (hereinafter, the amount of frequency spectrum shift Δf is referred to as ω) corresponding to the velocity of the blood flow is obtained as a Fourier spectrum.

The relationships shown in FIGS. 5 and 6 exist between: the shape of a Doppler spectrum; and the presence or absence of the blood vessel B and the velocity of the blood flow in the blood vessel B (a feature of the blood vessel). Specifically, when the blood vessel B does not exist in the area irradiated with the laser light L, because the above-described beat is not produced, the Doppler spectrum is formed into a flat shape having no intensity in the whole frequency ω region (see alternate long and short dashed line). When the blood vessel B with a slow blood flow exists therein, the Doppler spectrum has intensity in a region where the frequency ω is low and has a narrow spectral width (see solid line). When the blood vessel B with fast blood flow exists therein, the Doppler spectrum has intensity from a region where the frequency ω is low to a region where it is high and has a large spectral width (see dashed line). In this way, as the blood flow is increased, the Doppler spectrum expands toward a region where the frequency ω is high, thus increasing the spectral width, and the average frequency of the Doppler spectrum is increased accordingly.

Furthermore, it is known that the velocity of the blood flow in the blood vessel B is substantially proportional to the diameter of the blood vessel B (a feature of the blood vessel).

The frequency analysis unit 12 obtains a function F(ω), for a Doppler spectrum, showing the relationship between the frequency ω and the intensity, calculates the average frequency of a Doppler spectrum F(ω) on the basis of the following expression (1), and sends the calculated average frequency to the determination unit 13.

$$\begin{matrix}\{ Expression 1 \} & \\ Average Frequency = \frac{\int ω F ( ω ) d ω }{\int F ( ω ) d ω} & ( 1 )\end{matrix}$$

The determination unit 13 compares the average frequency received from the frequency analysis unit 12 with a threshold value and determines the presence or absence of the blood vessel B having a diameter that is within a predetermined diameter range, which serves as a predetermined diameter of blood vessel. The threshold value is the average frequency corresponding to the minimum value of the diameter of the detection-target blood vessel B. The determination unit 13 determines that the detection-target blood vessel B exists when the average frequency received from the frequency analysis unit 12 is equal to or greater than the threshold value. On the other hand, when the average frequency received from the frequency analysis unit 12 is less than the threshold value, the determination unit 13 determines that the detection-target blood vessel B does not exist in the area irradiated with the laser light L. Accordingly, a blood vessel B that has a diameter within the predetermined diameter range is set as a detection target, and the presence or absence of the detection-target blood vessel B is determined. The determination unit 13 outputs the determination result to the control unit 2.

The minimum value of the diameter of the detection-target blood vessel B is input by a surgeon, for example, using an input unit (not shown). The determination unit 13 has, for example, a function that associates the diameter of the blood vessel B with the average frequency, calculates, using the function, the average frequency associated with the input minimum value of the diameter of the blood vessel B, and sets the calculated average frequency as the threshold value.

When the determination unit 13 has determined that the detection-target blood vessel B does not exist, the control unit 2 causes the energy supply unit 5 to supply the energy source having a high intensity to the energy action portion 4, thereby actuating the energy action portion 4 in the incision mode. On the other hand, when the determination unit 13 has determined that the detection-target blood vessel B exists, the control unit 2 causes the energy supply unit 5 to supply the energy source having a lower intensity than the energy source used in the incision mode, to the energy action portion 4, thereby actuating the energy action portion 4 in the coagulation mode.

The frequency analysis unit 12, the determination unit 13, and the control unit 2 are realized by, for example, a computer that is provided with a central processing unit (CPU), a main storage unit, such as a RAM, and an auxiliary storage unit. The auxiliary storage unit is a non-temporal storage medium, such as a hard disk drive, and stores a program for causing the CPU to execute the processing of the above-described respective units 12, 13, and 2. When this program is loaded from the auxiliary storage unit into the main storage unit and is started, the CPU executes the processing of the respective units 12, 13, and 2 according to the program. Alternatively, the respective units 12, 13, and 2 may be realized by a PLD (programmable logic device) or an FPGA (field programmable gate array), or may be realized by dedicated hardware, such as an ASIC (application specific integrated circuit).

Next, the operation of the thus-configured surgical treatment device 100 will be described.

To treat the living tissue A by using the surgical treatment device 100 of this embodiment, a treatment target site of the living tissue A is gripped between the pair of jaws 6 and 7. The treatment target site between the jaws 6 and 7 is irradiated with the laser light L from the light emitting part 9, and the scattered light S of the laser light L that has been transmitted through the treatment target site while being scattered by the living tissue A is received by the light receiving part 10. The received scattered light S is detected by the light detection unit 11, and the time-series data of the scattered light S is generated in the frequency analysis unit 12. In the frequency analysis unit 12, the average frequency of a Doppler spectrum is extracted through frequency analysis of the time-series data, and the determination unit 13 determines whether a detection-target blood vessel B that has a diameter within the predetermined diameter range exists in the living tissue A, on the basis of the average frequency.

When it is determined that the detection-target blood vessel B does not exist in the treatment target site, the control unit 2 actuates the energy action portion 4 in the incision mode, thereby supplying high energy from the jaws 6 and 7 to the treatment target site and incising the treatment target site. When it is determined that detection-target blood vessel B exists in the treatment target site, the control unit 2 actuates the energy action portion 4 in the coagulation mode, thereby supplying low energy from the jaws 6 and 7 to the treatment target site and coagulating the treatment target site.

In this way, according to this embodiment, the Doppler shift of the scattered light S, which is caused by the blood flow in the blood vessel B, is analyzed, thereby detecting blood flowing in the blood vessel B while clearly distinguishing it from blood leaking from the blood vessel B due to bleeding. Accordingly, there is an advantage that it is possible to accurately detect the blood vessel B existing in the living tissue A. Furthermore, by using the fact that the shift Δf of the Doppler shift depends on the thickness of the blood vessel B, it is possible to recognize not only the presence or absence of the blood vessel B but also the thickness of the blood vessel B. Therefore, for example, there is an advantage that only a thick blood vessel B is detected by appropriately setting the threshold value, thus making it possible to appropriately control the actuation of the energy action portion 4 so as to reliably avoid incision of a treatment target site where the thick blood vessel B exists.

Note that, in this embodiment, when the determination unit 13 determines that the detection-target blood vessel B exists, the control unit 2 may display, for the surgeon, a sign indicating the existence of the detection-target blood vessel B, on a display unit (not shown), or may output a sound from a speaker (not shown). By doing so, the existence of the detection-target blood vessel B in the treatment target site can be reliably recognized by the surgeon.

Furthermore, in this embodiment, instead of controlling the intensity of the energy source to be supplied from the energy supply unit 5 to the energy action portion 4, the control unit 2 may allow the energy source to be supplied from the energy supply unit 5 to the energy action portion 4 when the determination unit 13 determines that the detection-target blood vessel B exists and may stop supplying the energy source from the energy supply unit 5 to the energy action portion 4 when the determination unit 13 determines that the detection-target blood vessel B does not exist.

By doing so, the action of the energy on the detection-target blood vessel B can be reliably avoided.

### Second Embodiment

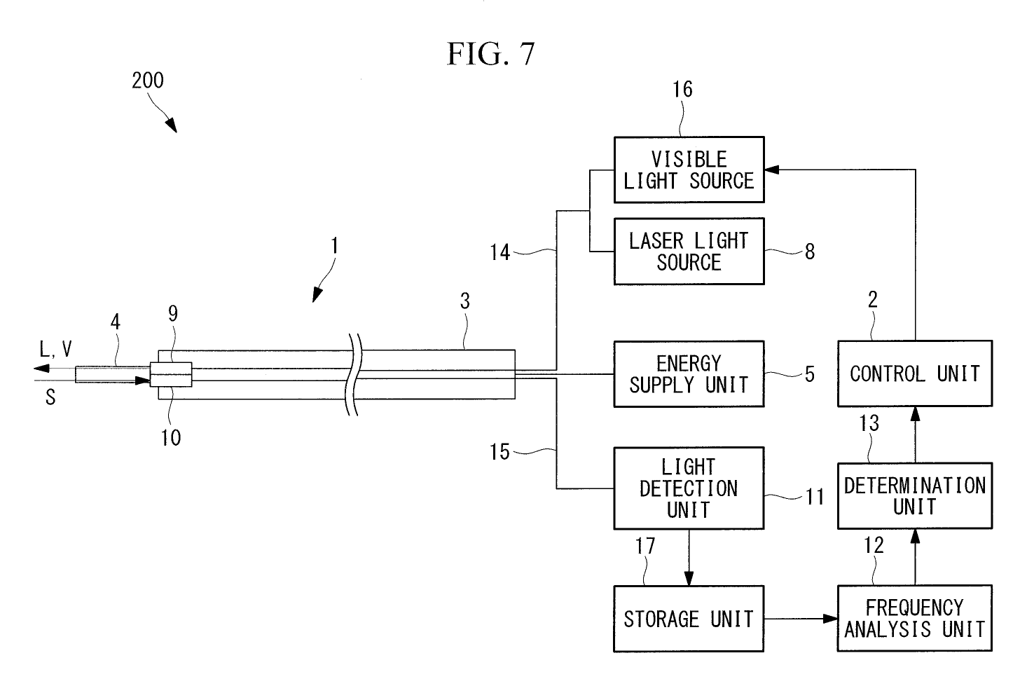

Next, a surgical treatment device 200 according to a second embodiment of the present invention will be described with reference to FIGS. 7 to 9.

In this embodiment, the following description will be mainly given for configurations that differ from those in the first embodiment, identical reference signs are assigned to configurations common to those in the first embodiment, and a description thereof will be omitted.

The surgical treatment device 200 of this embodiment mainly differs from that of the first embodiment in that the light emitting part 9 can radiate visible light V, in addition to the laser light L, onto the living tissue A, and the control unit 2 controls, not the energy action portion 4, but the light emitting part 9 to emit and not to emit the visible light V.

Specifically, as shown in FIG. 7, the blood-vessel detection means is further provided with a visible light source 16 that outputs visible light V having a wavelength in the visible wavelength region. It is preferred that the visible light source 16 be a laser light source. It is preferred that the color of the visible light V be a color with which the surgeon can easily view the visible light V radiated onto the living tissue A, for example, green or blue. The visible light V output from the visible light source 16 is combined with the laser light L output from the laser light source 8 by an optical system (not shown) and introduced to the irradiation optical fiber 14 together with the laser light L.

The light emitting part (visible-light radiating part) 9 is provided in the vicinity of the energy action portion 4 and emits the laser light L and the visible light V toward a front side of the distal end of the energy action portion 4.

The light receiving part 10 is provided in the vicinity of the light emitting part 9 and receives the scattered light S from the front side of the distal end of the energy action portion 4.

The determination unit 13 periodically repeats acquisition of the time-series data and periodically repeats determination of the presence or absence of the detection-target blood vessel B.

When the determination unit 13 determines that the detection-target blood vessel B exists, the control unit 2 causes the visible light source 16 to output the visible light V, thereby causing the light emitting part 9 to emit the visible light V, together with the laser light L. On the other hand, when the determination unit 13 determines that the detection-target blood vessel B does not exist, the control unit 2 causes the visible light source 16 to stop outputting the visible light V, thereby causing the light emitting part 9 to emit only the laser light L.

In this embodiment, the energy action portion 4 may be of any type other than the energy forceps.

The other configurations in this embodiment are the same as those in the first embodiment.

Next, the operation of the thus-configured surgical treatment device 200 will be described.

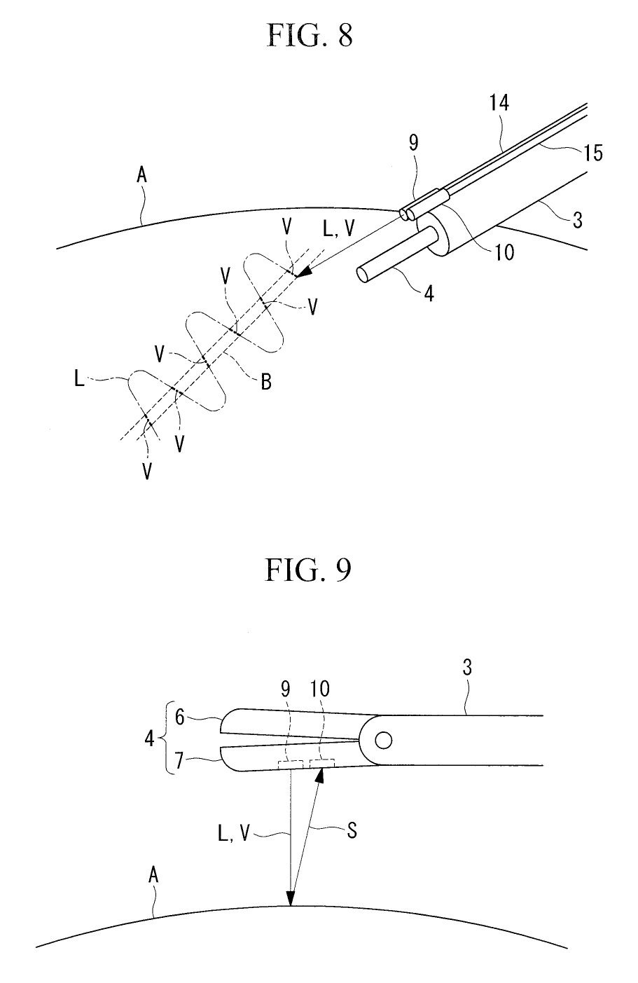

To treat the living tissue A by using the surgical treatment device 200 of this embodiment, the energy action portion 4 is disposed in the vicinity of the living tissue A, the laser light L is radiated from the light emitting part 9 onto the living tissue A, and the energy action portion 4 is moved so as to scan the laser light L on the living tissue A, as shown in FIG. 8. The scattered light S of the laser light L scattered by the living tissue A is received by the light receiving part 10. Thereafter, the presence or absence of the detection-target blood vessel B is determined as in the first embodiment.

When the determination unit 13 determines that the detection-target blood vessel B does not exist in the area irradiated with the laser light L, the control unit 2 causes the light emitting part 9 to emit only the laser light L. When the determination unit 13 determines that the detection-target blood vessel B exists in the area irradiated with the laser light L, the control unit 2 causes the light emitting part 9 to emit the visible light V together with the laser light L. Specifically, only when the detection-target blood vessel B exists in the area irradiated with the laser light L, the visible light V is also radiated onto this irradiated area.

Therefore, the surgeon can recognize that the area irradiated with the visible light V is an area where the detection-target blood vessel B exists. Accordingly, treatment of the living tissue A by using the energy action portion 4 is performed at a position other than the area irradiated with the visible light V, thereby making it possible to treat the living tissue A while reliably avoiding the detection-target blood vessel B. Because the advantageous effects of this embodiment are the same as those in the first embodiment, a description thereof will be omitted.

Note that, in this embodiment, the positions where the light emitting part 9 and the light receiving part 10 are attached to the energy treatment tool 1 may be changed depending on the type of the energy action portion 4.

For example, in a case in which the energy action portion 4 is energy forceps as in the first embodiment, as shown in FIG. 9, the light emitting part 9 and the light receiving part 10 may be provided on the outer surface of the lower jaw 7. The surgeon places the outer surface of the lower jaw 7 over the surface of the living tissue A and radiates the laser light L onto the living tissue A, thereby making it possible to check the presence or absence of the detection-target blood vessel B.

Furthermore, although this embodiment has the light paths of the laser light L and the visible light V in common, and the laser light L and the visible light V are radiated from the common light emitting part 9 onto the living tissue A, instead of this, a visible-light radiating part that is separate from the irradiation optical fiber 14 and the light emitting part 9 may be provided. The visible-light radiating part has a function of detecting a position irradiated with the laser light L, on the living tissue A, and is configured so as to be capable of radiating the visible light V onto the detected position irradiated with the laser light L.

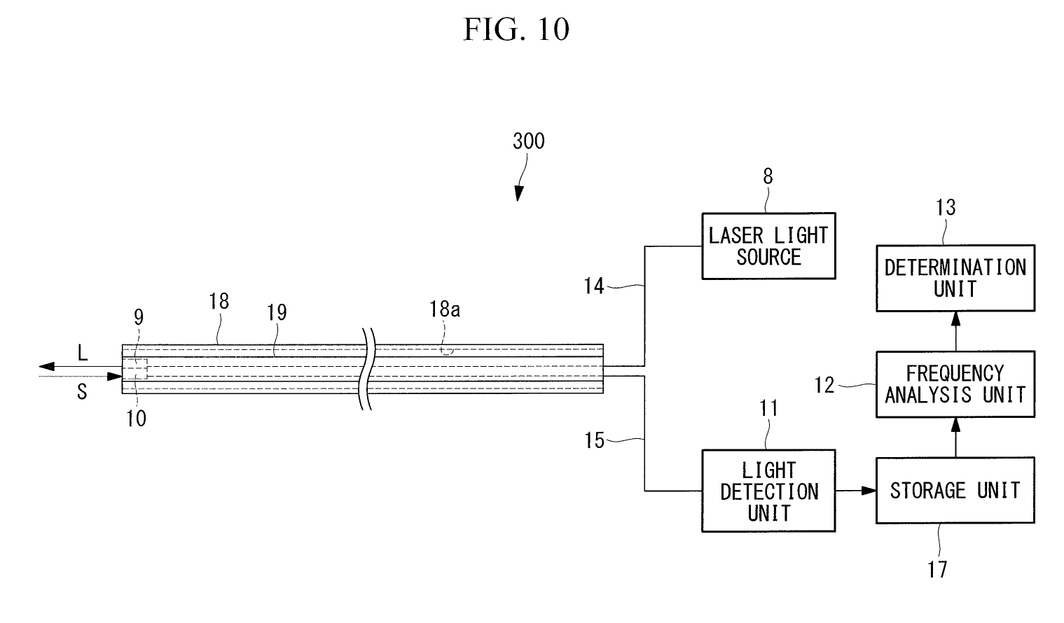

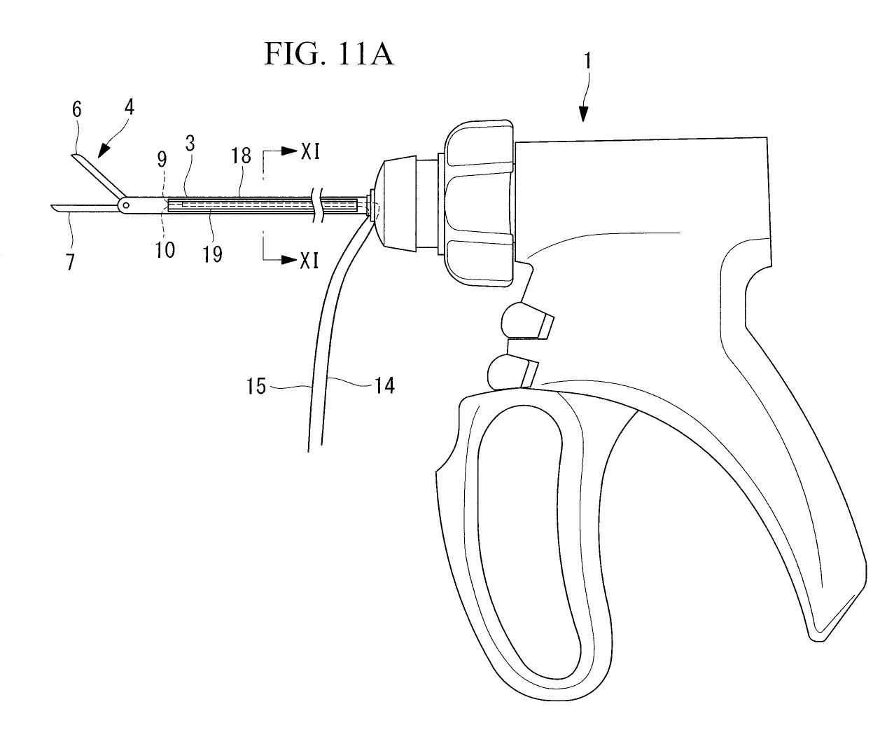

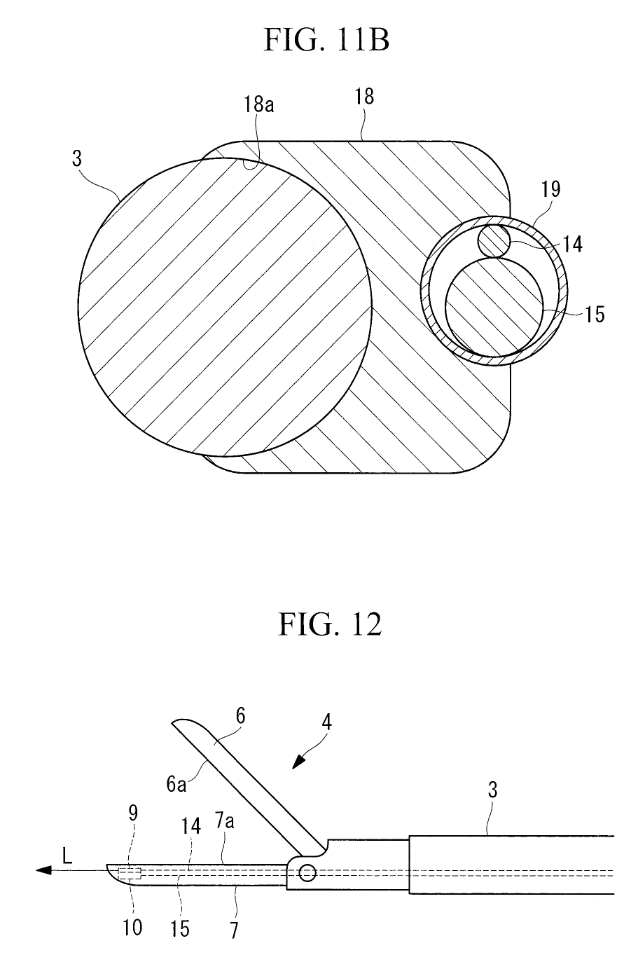

Next, a blood-vessel recognition device 300 according to a third embodiment of the present invention will be described with reference to FIGS. 10 to 11B.

In this embodiment, the following description will be mainly given for configurations that differ from those in the first embodiment, identical reference signs are assigned to the configurations common to those in the first embodiment, and a description thereof will be omitted.

The blood-vessel recognition device 300 of this embodiment is used by being attached to the energy treatment tool 1, and, as shown in FIG. 10, is provided with: an attachment portion 18 that holds the light emitting part 9 and the light receiving part 10 and that can be removably attached to the shaft portion 3 of the energy treatment tool 1; and a blood-vessel detection means. FIGS. 11A and 11B show a state in which the attachment portion 18 is attached to the shaft portion 3.

The attachment portion 18 is formed of an elongated columnar member made of an elastic material, and a fitting groove 18a that extends in the longitudinal direction is formed on an outer surface thereof. The fitting groove 18a has a substantially semicircular column shape having an inner diameter approximately equal to the outer diameter of the columnar shaft portion 3. The attachment portion 18 can be attached to an outer periphery of the shaft portion 3 by pressing the shaft portion 3 into the fitting groove 18a in a radial direction, and the attachment portion 18 can be removed from the shaft portion 3 by pulling out the shaft portion 3 from the inside of the fitting groove 18a in a radial direction.

A cylindrical sheath 19 is fixed, along the longitudinal direction, on an opposite side of the attachment portion 18 from the fitting groove 18a in the radial direction. The light emitting part 9, the light receiving part 10, the irradiation optical fiber 14, and the light-receiving optical fiber 15 are accommodated in the sheath 19, and the light emitting part 9 and the light receiving part 10 are disposed at a distal end portion of the sheath 19. The attachment portion 18 has a length dimension approximately equal to or less than the length dimension of the shaft portion 3 and can be attached to the shaft portion 3 so as to locate the light emitting part 9 and the light receiving part 10 in the vicinity of the energy action portion 4.

Although FIG. 10 shows the blood-vessel detection means similar to the blood-vessel detection means in the first embodiment, it is also possible to further provide the visible light source 16 and the control unit 2, which are explained in the second embodiment. Alternatively, when the determination unit 13 determines that the detection-target blood vessel B exists, a sign indicating that the detection-target blood vessel B exists may be displayed on a display unit (not shown) or a sound may be output from a speaker (not shown).

In this way, according to this embodiment, the blood-vessel recognition device 300 is provided separately from the energy treatment tool 1, thereby leading to an advantage that the blood-vessel recognition function can be added to the general-purpose energy treatment tool 1 as needed.

Note that, in the energy treatment tool 1 shown in FIGS. 11A and 11B, the light emitting part 9, the light receiving part 10, the irradiation optical fiber 14, and the light-receiving optical fiber 15 may be provided integrally with the energy treatment tool 1, thereby making it possible to configure a surgical treatment device. For example, as shown in FIG. 12, the light emitting part 9 and the light receiving part 10 may be provided at a distal end portion of the inside of the lower jaw 7, and the laser light L may be emitted from the distal end of the lower jaw 7 outward in the longitudinal direction of the shaft portion 3.

In the first to third embodiments, although the average frequency of a Doppler spectrum is used to determine the presence or absence of the blood vessel B and the diameter thereof, instead of this, the gradient of a Doppler spectrum or the spectral width may be used.

As shown in FIG. 5, the gradient of the Doppler spectrum is the amount of change ΔI in intensity between two predetermined frequencies ω1 and ω2. The differential value of the function F(ω) at a predetermined frequency ω may be used as the gradient of the Doppler spectrum. The predetermined frequencies ω1, ω2, and ω are set within a range allowing the gradient of a Doppler spectrum to gradually increase as the velocity of the blood flow increases from zero.

The spectral width is, for example, the half width W. As described above, the spectral width of the Doppler spectrum becomes larger as the blood flow becomes faster.

As with the average frequency, the gradient of the Doppler spectrum and the spectral width have a strong correlation with the velocity of the blood flow in the blood vessel B. Therefore, when the gradient or the spectral width is used instead of the average frequency, it is also possible to accurately estimate the amount of frequency spectrum shift Δf on the basis of the gradient or the spectral width, thereby making it possible to accurately figure out the presence or absence of the blood vessel B and the feature thereof (the blood-flow velocity or the blood-vessel diameter), and to determine the presence or absence of the detection-target blood vessel B with high accuracy.

Furthermore, in the first to third embodiments, although the energy action portion 4, which treats the living tissue A by using energy, is provided, the type of the action portion is not limited thereto and can be changed as appropriate. For example, the action portion may be an ordinary surgical knife.

Furthermore, in the first to third embodiments, it is preferred that the irradiation optical fiber 14 be a single-mode optical fiber, and the light-receiving optical fiber 15 be a multi-mode optical fiber. By doing so, the accuracy of recognition of the blood vessel B can be improved.

Specifically, to improve the accuracy of recognition of the blood vessel B, it is important to radiate stronger laser light L onto the blood vessel B, thus producing stronger scattered light S, and to collect, from a wider area, the scattered light S scattered from the blood vessel B in various directions, thus increasing the amount of received scattered light S. However, because the laser light L is scattered inside the living tissue A, the intensity of the laser light L rapidly decays.

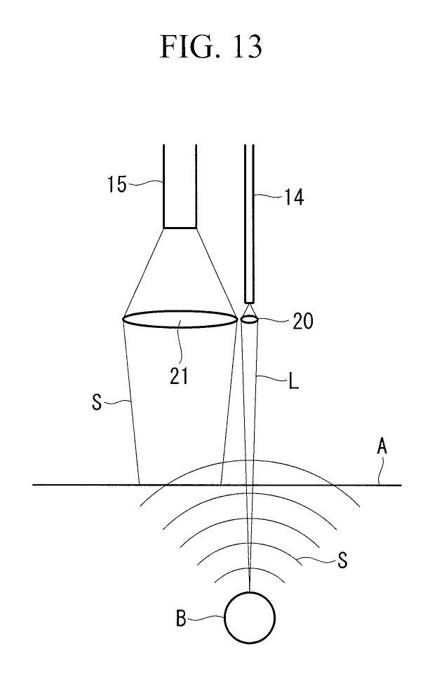

As shown in FIG. 13, as the irradiation optical fiber 14, a single-mode optical fiber that has a small core diameter and a small transmission cross-sectional area for the laser light L is used, thereby increasing the light density of the laser light L radiated from the irradiation optical fiber 14 onto the living tissue A. Accordingly, the laser light L can maintain a high intensity up to the blood vessel B, which is located inside the living tissue A, even when it is scattered by the living tissue A. Furthermore, a multi-mode optical fiber having a large core diameter is used as the light-receiving optical fiber 15, thereby making it possible to receive the scattered light S in a wider range.

Furthermore, it is preferred that a condenser lens 20 that has a short focal length so as to be in-focus in the vicinity of the surface of the living tissue A (for example, a position ±several millimeters away from the surface) and that converts the laser light L, which is diverging light, emitted from the irradiation optical fiber 14 into converging light be provided at the distal end of the irradiation optical fiber 14. By doing so, the light density of the laser light L in the living tissue A can be further increased.

Alternatively, a collimating lens having a short focal length may be provided at the distal end of the irradiation optical fiber 14. By doing so, the spot size of the laser light L becomes substantially constant even when the distance between the irradiation optical fiber 14 and the living tissue A fluctuates, thus making it possible to irradiate the living tissue A with the laser light L at a high light density independent of distance.

A light-receiving lens 21 that is separate from the condenser lens 20 may be provided at the distal end of the light-receiving optical fiber 15. The light-receiving lens 21 has a long focal length compared with that of the condenser lens 20.

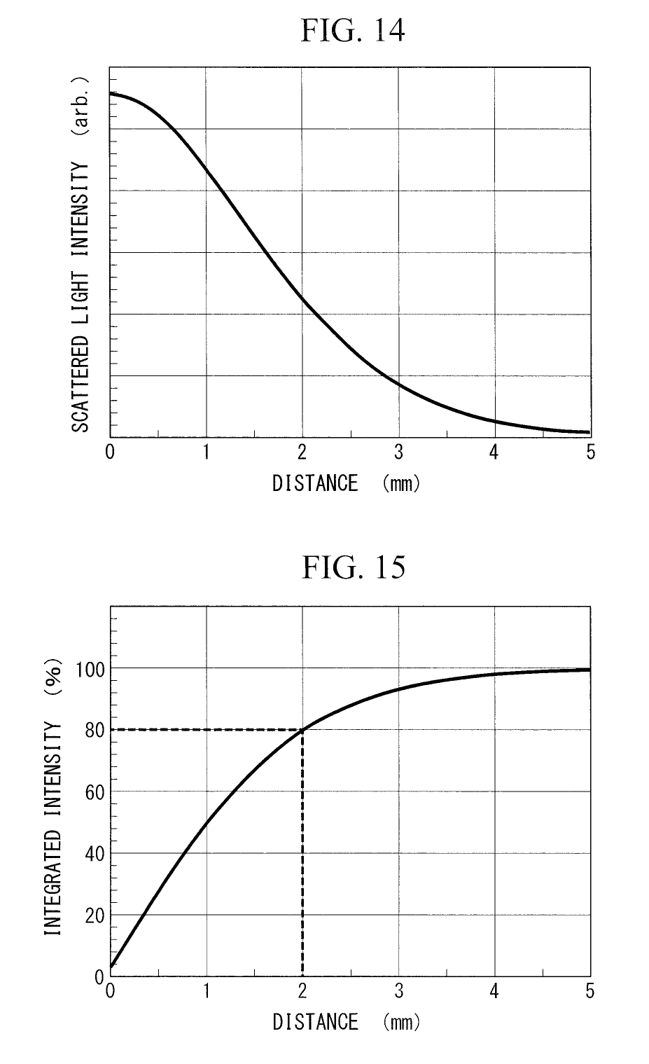

FIG. 14 shows the result obtained by simulating the spatial distribution, on a scatterer surface, of the intensity of scattered light produced in a blood vessel that is located at a depth of 3 mm from the surface of a scatterer corresponding to the living tissue A. Here, the distance indicated by the horizontal axis in FIG. 14 shows the distance above the scatterer surface when the position, on the scatterer surface, immediately above the center of the blood vessel in the vertical direction is the origin. FIG. 15 shows the integrated intensity of scattered light, obtained from the distribution shown in FIG. 14. In FIG. 15, the vertical axis is normalized such that the intensity obtained by integrating the intensities of scattered light from 0 mm to a sufficiently large distance is set to 100%.

The intensity of scattered light rapidly decays inside the scatterer, as shown in FIG. 14, and the integrated intensity in the range from the origin to 2 mm on the scatterer is 80%, as shown in FIG. 15. It is found from this result that the scattered light S in the range from the origin to 2 mm on the scatterer is received, thereby making it possible to ensure a sufficient amount of received scattered light S. Furthermore, even if the light-receiving range for the scattered light S is excessively expanded, a further increase in the amount of received light cannot be expected.

In this way, there is an appropriate size for the light-receiving range for the scattered light S. By providing the light-receiving lens 21, the light-receiving range for the scattered light S to be received by the light-receiving optical fiber 15 can be set to an appropriate size.

Furthermore, in the first to third embodiments, although the separate optical fibers 14 and 15 are used to transmit the laser light L and the scattered light S, respectively, instead of this, it is also possible to use a single double-clad fiber to transmit the laser light L and the scattered light S.

The double-clad fiber has a core, a first clad, and a second clad that are concentrically arranged in this order from a center side toward a radially outer side. The core and the first clad constitute a single-mode optical fiber that functions as the first transmission path, and the first clad and the second clad constitute a multi-mode optical fiber that functions as the second transmission path. Therefore, it is possible to transmit the laser light L by means of the core and the first clad and to transmit the scattered light S by means of the first clad and the second clad.

With this configuration, because the first transmission path and the second transmission path are coaxially provided, it is possible to perform, via a common lens, radiation of the laser light L onto the living tissue A and detection of the scattered light S from the living tissue A. Accordingly, it is possible to perform, with a simple structure, optical adjustment for locating the area irradiated with the laser light L within an area, in the living tissue A, where the scattered light S is detected and to increase the amount of received scattered light S.

The inventors have arrived at the following aspects of the present invention.

According to a first aspect, the present invention provides a blood-vessel recognition device including: a light emitting part that radiates laser light onto living tissue; a light receiving part that receives scattered light of the laser light scattered by the living tissue; a light detection unit that detects intensity of the scattered light received by the light receiving part; a frequency analysis unit that analyzes time-series data indicating a temporal change in the intensity of the scattered light detected by the light detection unit, to extract an amount of frequency spectrum shift of the scattered light, the amount of frequency spectrum shift being included in the time-series data; and a determination unit that determines a feature of a blood vessel in on the basis of the amount of frequency spectrum shift extracted by the frequency analysis unit.

According to the first aspect of the present invention, scattered light produced by radiating laser light onto living tissue from the light emitting part is received by the light receiving part, the intensity of the scattered light is detected by the light detection unit, and time-series data indicating a temporal change in the intensity of the scattered light is analyzed in the frequency analysis unit.

The frequency of the scattered light scattered by blood in a blood vessel, of the living tissue, is shifted with respect to the frequency of the laser light, due to the Doppler shift caused by the blood flow. The amount of frequency spectrum shift at this time has a correlation with a feature, such as blood-flow velocity or a blood-vessel diameter, of the blood vessel. On the other hand, the frequency of the scattered light scattered by components other than blood in a blood vessel, of the living tissue, is the same as the frequency of the laser light. Therefore, in a case in which a blood vessel does not exist in the living tissue, the intensity of the scattered light in the time-series data becomes substantially constant. On the other hand, in a case in which a blood vessel exists in the living tissue, the scattered light scattered by blood in the blood vessel and the scattered light scattered by components other than the blood vessel are simultaneously received by the light receiving part, thereby making a beat having the time period corresponding to the feature, such as blood-flow velocity or a blood-vessel diameter, of the blood vessel appear in the intensity of the scattered light in the time-series data.

In the frequency analysis unit, the amount of frequency spectrum shift corresponding to the presence or absence of a blood vessel and the feature, such as blood-flow velocity or a blood-vessel diameter, of the blood vessel is extracted from the time-series data. Therefore, in the determination unit, it is possible to accurately determine the presence or absence of a blood vessel while clearly distinguishing it from static blood, such as blood leaking from a blood vessel, and to determine the feature of the blood vessel existing in the living tissue, on the basis of the amount of frequency spectrum shift.

In the above-described first aspect, the frequency analysis unit may analyze the time-series data indicating the temporal change in the intensity of the scattered light, to extract an amount of frequency spectrum shift of the scattered light with respect to the laser light, the amount of frequency spectrum shift being included in the time-series data.

The above-described first aspect may further include a storage unit that stores, in a time series manner, the intensity of the scattered light detected by the light detection unit to generate the time-series data.

By doing so, the time-series data can be stored.

The above-described first aspect may further include: a visible-light radiating part that radiates visible light toward a position of the living tissue which is irradiated with the laser light; and a control unit that controls the visible-light radiating part to radiate the visible light onto the living tissue and not to radiate the visible light there, on the basis of a determination result obtained by the determination unit, wherein the control unit causes the visible-light radiating part to radiate the visible light onto the living tissue when the determination unit determines that a detection-target blood vessel having a diameter within a predetermined diameter range exists in the living tissue, and controls the visible-light radiating part not to radiate the visible light onto the living tissue when the determination unit determines that the detection-target blood vessel does not exist in the living tissue.

By doing so, only when the detection-target blood vessel exists in the area irradiated with the laser light, visible light is also radiated onto the irradiated area. Therefore, a user can recognize the area irradiated with the visible light as an area where the detection-target blood vessel exists.

In the above-described first aspect, the light emitting part may be able to radiate the visible light onto the living tissue together with the laser light to serves as the visible-light radiating part.

By doing so, with a simple structure, the position irradiated with the laser light can be accurately made to coincide with the position irradiated with the visible light.

In the above-described first aspect, the frequency analysis unit may obtain a Fourier spectrum by subjecting the time-series data to a Fourier transform and extracts, as the amount of frequency spectrum shift, an average frequency of the Fourier spectrum, a gradient thereof, or a spectral width thereof.

The velocity of the blood flow is substantially proportional to the square of the diameter of the blood vessel, and the average frequency of the Fourier spectrum, the gradient, and the spectral width have a strong correlation with the velocity of the blood flow. Therefore, it is possible to accurately figure out the amount of frequency spectrum shift on the basis of the average frequency, the gradient, or the spectral width, and to improve the accuracy of determination of the presence or absence of a blood vessel and the feature thereof performed by the determination unit.

The above-described first aspect may further include an attachment portion that holds the light emitting part and the light receiving part and that can be removably attached to a treatment tool. The attachment portion may be removably attached to a shaft portion of the treatment tool.

By doing so, the blood-vessel recognition device is integrated with the treatment tool, thus making it possible to manipulate the blood-vessel recognition device and the treatment tool together.

The above-described first aspect may further include: a first transmission path that transmits the laser light to the light emitting part; and a second transmission path that transmits the scattered light from the light receiving part to the light detection unit and that is different from the first transmission path, wherein a transmission cross-sectional area for the laser light of the first transmission path is smaller than a transmission cross-sectional area for the scattered light of the second transmission path. The first transmission path may transmit the laser light in a single mode; and the second transmission path may transmit the scattered light in a multiple mode.

By doing so, laser light having a high light density is radiated onto the living tissue from the first transmission path, and thus the laser light having a high intensity acts on a deep position in the living tissue as well. Accordingly, strong scattered light can be produced. Furthermore, because the scattered light in a wide range is received by the second transmission path, the amount of received scattered light can be increased.

In the above-described first aspect, the first transmission path may be formed of a core and a first clad of a double-clad fiber, and the second transmission path may be formed of the first clad and a second clad of the double-clad fiber.

A double-clad fiber has a single core, a first clad, and a second clad, and the core, the first clad, and the second clad are concentrically arranged in this order from the center toward a radially outer side. The core and the first clad have a function equivalent to that of a single-mode optical fiber, and the first clad and the second clad have a function equivalent to that of a multi-mode optical fiber.

Therefore, the core and the first clad are used as the first transmission path, and the first clad and the second clad are used as the second transmission path, thereby coaxially configuring the first transmission path and the second transmission path. Accordingly, it is possible to perform, with a simple structure, optical adjustment for locating the area irradiated with the laser light within an area, in the living tissue, where the scattered light is detected and to increase the amount of received scattered light.

According to a second aspect, the present invention provides a surgical treatment device including: an action portion that treats living tissue; a light emitting part that is provided in the action portion or in a vicinity of the action portion and that radiates laser light onto the living tissue; a light receiving part that receives scattered light of the laser light scattered by the living tissue; a light detection unit that detects intensity of the scattered light received by the light receiving part; a frequency analysis unit that analyzes time-series data indicating a temporal change in the intensity of the scattered light detected by the light detection unit, to extract an amount of frequency spectrum shift of the scattered light, the amount of frequency spectrum shift being included in the time-series data; and a determination unit that determines presence or absence of a blood vessel with the predetermined specific range of the diameter in the living tissue on the basis of the amount of frequency spectrum shift extracted by the frequency analysis unit.

In the above-described second aspect, the frequency analysis unit may analyze the time-series data indicating a temporal change in the intensity of the scattered light, to extract an amount of frequency spectrum shift of the scattered light with respect to the laser light, the amount of frequency spectrum shift being included in the time-series data.

In the above-described second aspect, the action portion may be an energy action portion that causes energy to act on the living tissue, the above-described second aspect may further include: an energy supply unit that supplies, to the energy action portion, an energy source for generating the energy; and a control unit that controls the energy supply unit on the basis of a determination result obtained by the determination unit.

By doing so, it is possible to switch the operation of the energy action portion depending on whether the detection-target blood vessel exists in the living tissue.

In the above-described second aspect, the control unit may control the energy supply unit not to supply the energy source to the energy action portion when the determination unit determines that a detection-target blood vessel having a diameter within a predetermined diameter range exists in the living tissue.

By doing so, only when the detection-target blood vessel does not exist, energy can be selectively caused to act on the living tissue from the energy action portion to perform treatment.

In the above-described second aspect, the control unit may switch an intensity control mode for the energy source supplied from the energy supply unit to the energy action portion when the determination unit determines that a detection-target blood vessel having a diameter within a predetermined diameter range exists in the living tissue.

By doing so, it is possible to treat the living tissue by using an incision mode when the detection-target blood vessel does not exist and by using a coagulation mode when the detection-target blood vessel exists in the living tissue.

The above-described second aspect may further include: a visible-light radiating part that radiates visible light toward a position of the living tissue which is irradiated with the laser light; and a control unit that controls the visible-light radiating part to radiate the visible light onto the living tissue and not to radiate the visible light there, on the basis of a determination result obtained by the determination unit, wherein the control unit causes the visible-light radiating part to radiate the visible light onto the living tissue when the determination unit determines that a detection-target blood vessel having a diameter within a predetermined diameter range exists in the living tissue, and controls the visible-light radiating part not to radiate the visible light onto the living tissue when the determination unit determines that the detection-target blood vessel does not exist in the living tissue.

In the above-described second aspect, the light emitting part may be able to radiate the visible light onto the living tissue together with the laser light to serves as the visible-light radiating part.

In the above-described second aspect, the frequency analysis unit may obtain a Fourier spectrum by subjecting the time-series data to a Fourier transform and extracts, as the amount of frequency spectrum shift, an average frequency of the Fourier spectrum, a gradient thereof, or a spectral width thereof.

The above-described second aspect may further include: a first transmission path that transmits the laser light to the light emitting part; and a second transmission path that transmits the scattered light from the light receiving part to the light detection unit and that is different from the first transmission path, wherein a transmission cross-sectional area for the laser light of the first transmission path is smaller than a transmission cross-sectional area for the scattered light of the second transmission path.

The first transmission path may transmit the laser light in a single mode; and the second transmission path may transmit the scattered light in a multiple mode. Furthermore, the first transmission path may be formed of a core and a first clad of a double-clad fiber, and the second transmission path may be formed of the first clad and a second clad of the double-clad fiber.

### Advantageous Effects of Invention

According to the aforementioned aspects of the present invention, an advantageous effect is afforded in that a blood vessel existing in living tissue can be accurately detected, and a blood vessel having a predetermined feature, such as blood-flow velocity or a blood-vessel diameter, can be selectively detected.

### Reference Signs List

1 energy treatment tool
2 control unit
3 shaft portion
4 energy action portion (action portion)
5 energy supply unit
6, 7 jaw
6a, 7a inner surface
8 laser light source
9 light emitting part (visible-light radiating part)
10 light receiving part
11 light detection unit
12 frequency analysis unit
13 determination unit
14 irradiation optical fiber (first transmission path)
15 light-receiving optical fiber (second transmission path)
16 visible light source
17 storage unit
18 attachment portion
18a fitting hole
19 sheath
20 condenser lens
21 light-receiving lens
100, 200 surgical treatment device
300 blood-vessel recognition device
L laser light
S scattered light
V visible light
A living tissue
B blood vessel
C red blood cell

1. A blood-vessel recognition device comprising:
a light emitting part that radiates laser light onto living tissue;
a light receiving part that receives scattered light of the laser light scattered by the living tissue;
a light detection unit that detects intensity of the scattered light received by the light receiving part;
a frequency analysis unit that analyzes time-series data indicating a temporal change in the intensity of the scattered light detected by the light detection unit, to extract an amount of frequency spectrum shift of the scattered light, the amount of frequency spectrum shift being included in the time-series data; and
a determination unit that determines a feature of a blood vessel in on the basis of the amount of frequency spectrum shift extracted by the frequency analysis unit.

2. The blood-vessel recognition device according to claim 1, wherein the frequency analysis unit analyzes the time-series data indicating the temporal change in the intensity of the scattered light, to extract an amount of frequency spectrum shift of the scattered light with respect to the laser light, the amount of frequency spectrum shift being included in the time-series data.

3. The blood-vessel recognition device according to claim 1 or 2, further comprising a storage unit that stores, in a time series manner, the intensity of the scattered light detected by the light detection unit to generate the time-series data.

4. The blood-vessel recognition device according to claim 1, further comprising:
a visible-light radiating part that radiates visible light toward a position of the living tissue which is irradiated with the laser light; and
a control unit that controls the visible-light radiating part to radiate the visible light onto the living tissue and not to radiate the visible light there, on the basis of a determination result obtained by the determination unit,
wherein the control unit causes the visible-light radiating part to radiate the visible light onto the living tissue when the determination unit determines that a detection-target blood vessel having a diameter within a predetermined diameter range exists in the living tissue, and controls the visible-light radiating part not to radiate the visible light onto the living tissue when the determination unit determines that the detection-target blood vessel does not exist in the living tissue.

5. The blood-vessel recognition device according to claim 4, wherein the light emitting part can radiate the visible light onto the living tissue together with the laser light to serves as the visible-light radiating part.

6. The blood-vessel recognition device according to claim 1, wherein the frequency analysis unit obtains a Fourier spectrum by subjecting the time-series data to a Fourier transform and extracts, as the amount of frequency spectrum shift, an average frequency of the Fourier spectrum, a gradient thereof, or a spectral width thereof.

7. The blood-vessel recognition device according to claim 1, further comprising an attachment portion that holds the light emitting part and the light receiving part and that can be removably attached to a treatment tool.

8. The blood-vessel recognition device according to claim 7,
wherein the treatment tool is provided with: an elongated shaft portion; and an action portion that is provided at a distal end of the shaft portion and that treats the living tissue; and
wherein the attachment portion can be removably attached to the shaft portion of the treatment tool.

9. The blood-vessel recognition device according to claim 1, further comprising:
a first transmission path that transmits the laser light to the light emitting part; and
a second transmission path that transmits the scattered light from the light receiving part to the light detection unit and that is different from the first transmission path,
wherein a transmission cross-sectional area for the laser light of the first transmission path is smaller than a transmission cross-sectional area for the scattered light of the second transmission path.

10. The blood-vessel recognition device according to claim 9,
wherein the first transmission path transmits the laser light in a single mode; and
the second transmission path transmits the scattered light in a multiple mode.

11. A surgical treatment device comprising:
an action portion that treats living tissue;
a light emitting part that is provided in the action portion or in a vicinity of the action portion and that radiates laser light onto the living tissue;
a light receiving part that receives scattered light of the laser light scattered by the living tissue;
a light detection unit that detects intensity of the scattered light received by the light receiving part;
a frequency analysis unit that analyzes time-series data indicating a temporal change in the intensity of the scattered light detected by the light detection unit, to extract an amount of frequency spectrum shift of the scattered light, the amount of frequency spectrum shift being included in the time-series data; and
a determination unit that determines a feature of a blood vessel in the living tissue on the basis of the amount of frequency spectrum shift extracted by the frequency analysis unit.

12. The surgical treatment device according to claim 11, wherein the frequency analysis unit analyzes the time-series data indicating the temporal change in the intensity of the scattered light, to extract an amount of frequency spectrum shift of the scattered light with respect to the laser light, the amount of frequency spectrum shift being included in the time-series data.

13. The surgical treatment device according to claim 11,
wherein the action portion is an energy action portion that causes energy to act on the living tissue,
the surgical treatment device further comprising:
an energy supply unit that supplies, to the energy action portion, an energy source for generating the energy; and
a control unit that controls the energy supply unit on the basis of a determination result obtained by the determination unit.

14. The surgical treatment device according to claim 13, wherein the control unit controls the energy supply unit not to supply the energy source to the energy action portion when the determination unit determines that a detection-target blood vessel having a diameter within a predetermined diameter range exists in the living tissue.

15. The surgical treatment device according to claim 13, wherein the control unit switches an intensity control mode for the energy source supplied from the energy supply unit to the energy action portion when the determination unit determines that a detection-target blood vessel having a diameter within a predetermined diameter range exists in the living tissue.

16. The surgical treatment device according to claim 11, further comprising:
a visible-light radiating part that radiates visible light toward a position of the living tissue which is irradiated with the laser light; and
a control unit that controls the visible-light radiating part to radiate the visible light onto the living tissue and not to radiate the visible light there, on the basis of a determination result obtained by the determination unit,
wherein the control unit causes the visible-light radiating part to radiate the visible light onto the living tissue when the determination unit determines that a detection-target blood vessel having a diameter within a predetermined diameter range exists in the living tissue, and controls the visible-light radiating part not to radiate the visible light onto the living tissue when the determination unit determines that the detection-target blood vessel does not exist in the living tissue.

17. The surgical treatment device according to claim 16, wherein the light emitting part can radiate the visible light onto the living tissue together with the laser light to serves as the visible-light radiating part.

18. The surgical treatment device according to claim 11, wherein the frequency analysis unit obtains a Fourier spectrum by subjecting the time-series data to a Fourier transform and extracts, as the amount of frequency spectrum shift, an average frequency of the Fourier spectrum, a gradient thereof, or a spectral width thereof.

19. The surgical treatment device according to claim 11, further comprising:
a first transmission path that transmits the laser light to the light emitting part; and
a second transmission path that transmits the scattered light from the light receiving part to the light detection unit and that is different from the first transmission path,
wherein a transmission cross-sectional area for the laser light of the first transmission path is smaller than a transmission cross-sectional area for the scattered light of the second transmission path.

20. The surgical treatment device according to claim 19,
wherein the first transmission path transmits the laser light in a single mode; and
the second transmission path transmits the scattered light in a multiple mode.
